(19) United States
(12) Patent Application Publication
(10) Pub. No.: US 2004/0185029 A1
(43) Pub. Date: Sep. 23, 2004

(54) Polymeric drug

(76) Inventor: Haizhong Tang, Tuscaloosa, AL (US)

Correspondence Address: Donald W. Meeker, Patent Agent, 924 East Ocean Front #E, Newport Beach, CA, 92661, US

(21) Appl. No.: 10/392,711

(22) Filed: Mar. 19, 2003

(51) Int. Cl. A61K 31/765; C08G 69/26

(52) U.S. Cl. 424/78.37; 528/348

(57) ABSTRACT

Condensing small drug molecules which contain an acid and alcohol, thiol, or phenol, or amine groups to polymerize them with a diimidazole reagent or thionyl chloride-pyridine complex to form a drug polymer which is a polyester or a polyamide in a one-step reaction. Carbonyldiimidazole, or thiocarbonyldiimidazole, or thionyl chloride-pyridine complex, or oxalyl chloride-pyridine complex or any other similar diimidazole, or pyridine reagents are used to condense salicyclic acid itself into a polymer, the polyester polysalicyclic acid. A one-step reaction synthesizes the exclusively active drug ingredients so that the polymers produced consist only of the drugs themselves, the target molecules, thus providing a drug delivery efficiency of 100%.

### BACKGROUND OF THE INVENTION

1. Field of the Invention

The present invention relates to the field of polymeric drugs, and in particular a method of preparing said drugs in a one step reaction.

2. Description of the Prior Art

The field of polymerizing drugs is well known, in most cases the method of linking the drug to the polymer involves many steps and often using inactive linkers which are not part of the healing process so that the efficiency of drug delivery is reduced.

Prior art U.S. Pat. No. 6,486,214, issued Nov. 26, 2002 to Uhrich, provides polyanhydrides that link low molecular weight drugs containing a carboxylic acid group and an amine, thiol, alcohol or phenol group within their structure into polymeric drug delivery systems are provided. Also provided are methods of producing polymeric drug delivery systems via these polyanhydride linkers as well as methods of administering low molecular weight drugs to a host via the polymeric drug delivery systems.

Prior art U.S. Pat. No. 6,468,519, issued Oct. 22, 2002 to Uhrich, shows polyanhydrides that degrade into biologically active salicylates and alpha-hydroxy acids and methods of using these polyanhydrides to deliver the salicylates and alpha-hydroxy acids to a host are provided.

Prior art U.S. Pat. No. 6,339,067, issued Jan. 15, 2002 to Wolff, claims a method of forming polymers in the presence of nucleic acid using template polymerization. Also, a method that has the polymerization occur in heterophase systems. These methods can be used for the delivery of nucleic acids, for condensing the nucleic acid, for forming nucleic acid binding polymers, for forming supramolecular complexes containing nucleic acid and polymer, and for forming an interpolyelectrolyte complex.

Prior art U.S. Pat. No. 5,738,864, issued Apr. 14, 1998 to Schacht, describes a conjugate of a carrier polymer and aziridine ring containing mitomycin (MMC) drug molecules that is prepared by coupling the MMC molecules via their aziridine imino groups to spacer groups that terminate in protected amino groups, deprotecting said amino groups, recovering and purifying the spacer-MMC derivatives, and then coupling these derivatives via said deprotected amino groups to the carrier polymer. Alternatively, the MMC may first be treated with an activating agent, e.g. carbodiimidazole, to form an activated MMC derivative that is then coupled directly to spacer groups linked to the carrier polymer.

Prior art U.S. Pat. No. 5,648,506, issued Jul. 15, 1997 to Desai, discloses polymeric drug delivery systems in which the drug is bound to a water-soluble polymer to provide a form of soluble drug delivery especially for those cases in which the drug by itself is water-insoluble. In particular, the drug taxol is covalently bound to water-soluble polyethylene glycols such as linear polyethylene glycols, branched polyethylene glycols, star polyethylene glycols, and branched copolymers of polyethylene glycols with other functional monomers to comprise a form of polymeric drug delivery. Also, crosslinked insoluble gels of these materials are prepared to serve as a form of implantable drug delivery.

Prior art U.S. Pat. No. 6,150,341, issued Nov. 21, 2000 to Russell-Jones, indicates methods for preparing vitamin B.sub.12 (VB.sub.12) derivatives suitable for linking to a polymer, nanoparticle or therapeutic agent, protein or peptide. The methods involve reacting the 5′OH group of VB.sub.12 or an analogue thereof with an active carbonyl electrophile and subsequently obtaining said VB.sub.12 derivatives. The invention also relates to novel VB.sub.12 derivatives, VB.sub.12 derivatives prepared by the methods of the present invention and uses thereof in the preparation of in the preparation of polymer complexes or nanoparticles.

Prior art U.S. Pat. No. 6,126,964, issued Oct. 3, 2000 to Wolff, concerns a method of forming polymers in the presence of nucleic acid using template polymerization. Also, a method that has the polymerization occur in heterophase systems. These methods can be used for the delivery of nucleic acids, for condensing the nucleic acid, for forming nucleic acid binding polymers, for forming supramolecular complexes containing nucleic acid and polymer, and for forming an interpolyelectrolyte complex.

Prior art U.S. Pat. No. 4,999,417, issued Mar. 12, 1991 to Domb, illustrates biodegradable polyanhydrides or polyester compositions based on amino acids. The compositions may be used as carriers for drugs or the like or as the drug source itself. The polymers are prepared from amino acids that are modified to include an additional carboxylic acid group.

Prior art U.S. patent application No. 20020177680, issued Nov. 28, 2002 to Hubbell, is for a composition that comprises a pre-formed, hydrolytically susceptible non-addition polyanionic polymer. The polymer comprises polymer strands formed from at least one ethylenically unsaturated monomer and links the polymer strands by at least one linking moiety comprising a hydrolytically susceptible bond, wherein at least one of which monomers has: a) one or more functional groups that can be titrated with base to form negatively charged functional groups; or b) one or more precursor groups that are precursors of the functional groups that can be titrated with base; which precursor groups are converted to the functional groups.

Prior art U.S. patent application No. 20020160109, issued Oct. 31, 2002 to Yeo, a solvent exchange method that is employed to provide microencapsulated compositions, such as microcapsules of pharmaceutical preparations. The method is based on an exchange of water and a hydrophilic organic solvent, whereby a decline in solvent quality for the organic solvent causes a polymer dissolved therein to be deposited onto an aqueous core. Optimal results are rationalized in terms of a balance of water solubility and surface tension for the organic solvent. In a preferred embodiment, microcapsules of selected drugs are formed by contacting microdroplets of an aqueous solution containing the drug with the organic solvent containing a polymer dissolved therein. A preferred method employs biodegradable poly(lactic acid-co-glycolic acid) (PLGA) dissolved in acetic acid, ethyl acetate, methyl acetate, or ethyl formate, to form a PLGA membrane around an aqueous drug core. The method is particularly attractive for encapsulating protein-based drugs without substantial denaturation.

Prior art U.S. patent application No. 20030012734, issued Jan. 16, 2003 to Pathak, shows biocompatible crosslinked polymers, and methods for their preparation and use. The biocompatible crosslinked polymers are formed from water soluble precursors that have electrophilic and nucleophilic functional groups capable of reacting and crosslinking in situ. Methods for making the resulting biocompatible crosslinked polymers biodegradable or not are provided, as are methods for controlling the rate of degradation. The crosslinking reactions may be carried out in situ on organs or tissues or outside the body. Applications for such biocompatible crosslinked polymers and their precursors include controlled delivery of drugs, prevention of post-operative adhesions, coating of medical devices such as vascular grafts, wound dressings and surgical sealants. Visualization agents may be included with the crosslinked polymers.

Prior art U.S. patent application No. 20020136769, issued Sep. 26, 2002 to Kabanov, depicts nanogel networks that have at least one cross-linked polyionic polymer fragment and at least one nonionic water-soluble polymer fragment, and compositions thereof, that have at least one suitable biological agent.

Prior art U.S. Pat. No. 5,420,105, issued May 30, 1995 to Gustavson, describes polymeric carriers that are polypeptides which comprise at least one drug-binding domain that non-covalently binds a drug. A polymeric carrier may be attached to an antibody specific for desired target cells to form immunoconjugates that deliver a drug to the target cells in vivo. A polymeric carrier may be attached to a proteinaceous or a non-proteinaceous ligand or anti-ligand to form a conjugate useful in pretargeting protocols to deliver a drug to target cells in vivo. The carriers are derived from drug-binding proteins and produced through peptide synthesis or recombinant DNA technology.

Prior art U.S. Pat. No. 6,150,472, issued Nov. 21, 2000 to Engbers, discloses polymers that have multi-functional sites and a gel comprising a solvent swollen network of cross-linked polymer(s), of which at least one polymer comprises at least one multi-functional site. A multi-functional site is a sequence of more than one functional group. A multi-functional polymer is a polymer comprising one or more multi-functional sites and/or more than one functional group.

Prior art U.S. Pat. No. 6,333,051, issued Dec. 25, 2001 to Kabanov, indicates copolymer networks that have at least one cross-linked polyamine polymer fragment and at least one nonionic water-soluble polymer fragment, and compositions thereof, which have at least one suitable biological agent.

What is needed is a one-step method for making drug polymers using only beneficial drug ingredients.

### SUMMARY OF THE INVENTION

An object of the present invention is to synthesize polymeric drugs using only a one step reaction to get the target molecules.

Another object of the present invention is to use carbonyldiimidazole, or thiocarbonyldiimidazole, or thionyl chloride-pyridine complex, or other similar reagents to condense salicyclic acid itself into a polymer which is the polyester polysalicyclic acid.

Yet another object of the present invention is to condense any small molecular drugs which contain carboxylic acid and hydroxyl or amine groups to polymerize them into a polyester or a polyamide in a one-step reaction by using a diimidazole reagent, wherein the drug molecules themselves are interconnected into a polymer.

One more object of the present invention is to use only the drugs themselves in the reactions without introducing inactive linkers, which are useless to treatment, so that the polymers produced consist only of the useful drugs themselves, providing a 100% efficiency of drug delivery.

In brief, small bioactive molecules having at least one acid group and at least one alcohol, or one phenol, or one thiol, or one amine group have now been successfully polymerized by a one-step reaction. In one embodiment, salicyclic acid is condensed to the poly(salicyclic acid) ester in high yield for the first time. In another embodiment, para-aminosalicyclic acid is also polymerized in high yield. Polyesters and polyamides are well-known biodegradable drug delivery carriers. Salicyclic acid is the active ingredient of Aspirin in the human body. Aspirin is one of the world's safest and least expensive pain relievers with over 100 years of proven usage. It is also effective in arthritis and preventing heart attack and strokes. It is the active ingredient in more than 50 over-the-counter medications. Newest studies also show that aspirin can reduce the risk of many cancers. However, prolonged use of aspirin can result in stomach bleeding and ulcers. Unlike regular aspirin, the structure of the polyester of salicyclic acid allows it to survive this acid environment in the stomach, thus reducing Aspirin's side affects.

Carbonyldiimidazole, or thiocarbonyldiimidazole, or other similar reagents are used to condense salicyclic acid itself into polymers, so the polymers produced are polyesters (polysalicyclic acid). A one-step reaction synthesizes the exclusively active drug ingredients so that the polymers produced consist only of the drugs themselves, the target molecules, thus providing a drug delivery efficiency of 100%.

Small drug molecules which contain carboxylic acid and hydroxyl or amine groups can be polymerized by carbonyldiimidazole. For example, salicylic acid, which is the active form of aspirin, can be prepared in polymer form, (polysalicylic acid) in a one step reaction.

Many other small molecular drugs which contain carboxylic acid and hydroxyl or amine groups can be polymerized by using a diimidazole reagent. In the present invention, the drug molecules themselves are interconnected into a polymer. Many small molecules which contain carboxylic acid and hydroxyl or amine groups can be polymerized by using this method.

An advantage of the present invention is that it requires only one step.

Another advantage of the present invention is that it produces a drug delivery efficiency of 100%.

### BEST MODE FOR CARRYING OUT THE INVENTION

Small bioactive molecules having at least one acid group and at least one alcohol, or one phenol, or one thiol, or one amine group have now been successfully polymerized by a one-step reaction. In one embodiment, salicyclic acid is condensed to the poly(salicyclic acid) ester in high yield for the first time. In another embodiment, para-aminosalicyclic acid is also polymerized in high yield. Polyesters and polyamides are well-known biodegradable drug delivery carriers. Salicyclic acid is the active ingredient of Aspirin in the human body. Aspirin is one of the world's safest and least expensive pain relievers with over 100 years of proven usage. It is also effective in arthritis and preventing heart attack and strokes. It is the active ingredient in more than 50 over-the-counter medications. Newest studies also show that aspirin can reduce the risk of many cancers. However, prolonged use of aspirin can result in stomach bleeding and ulcers. Unlike regular aspirin, the structure of the polyester of salicyclic acid allows it to survive this acid environment in the stomach, thus reducing Aspirin's side affects.

A method for making polymerized drugs in a one-step process comprises condensing small drug molecules which contain an acid and an alcohol, or an thiol, or phenol or amine groups to polymerize them with a diimidazole reagent or thionyl chloride-pyridine complex, or oxalyl chloride-pyridine complex or any other similar acylating reagent to form a drug polymer which is a polyester or a polyamide in a one-step reaction.

The method for making polymerized drugs in a one-step process may comprise the single step of polymerizing salicylic acid with thionyl chloride-pyridine complexes to produce the polyester, poly(salicylic acid).

The method for making polymerized drugs in a one-step process may comprise the single step of polymerizing 4-amino-salicyclic acid with carbonyldiimidazole to produce the polyamide, poly(4-amino-salicyclic acid).

The method for making polymerized drugs in a one-step process may comprise the single step of condensing salicylic acid with thiocarbonyldiimidazole or other similar acylating reagents to produce the polymer poly(salicylic acid).

The method for making polymerized drugs in a one-step process may comprise using thionyl-pyridine complex, oxalyl chloride-pyridine complex, in which pyridine could be pyridine itself, or any other substituted pyridine derivatives.

The method for making polymerized drugs in a one-step process may comprise using at least one acid group in the monomers.

The method for making polymerized drugs in a one-step process wherein the acid group is a group taken from a list of acid groups including carboxylic acid, sulfonic acid, sulfinic acid, phosphonic acid, or any other acid group.

Carbonyldiimidazole, or thiocarbonyldiimidazole, or thionyl chloride-pyridine complex, or other similar reagents are used to condense salicyclic acid itself into polymers, so the polymers produced are polyesters (polysalicyclic acid). A one-step reaction synthesizes the exclusively active drug ingredients so that the polymers produced consist only of the drugs themselves, the target molecules, thus providing a drug delivery efficiency of 100%.

Small drug molecules which contain carboxylic acid and hydroxyl or amine groups can be polymerized by carbonyldiimidazole. For example, salicylic acid, which is the active form of aspirin, can be prepared in polymer form, (polysalicylic acid) in a one step reaction.

Many other small molecular drugs which contain carboxylic acid and hydroxyl or amine groups can be polymerized by using diimidazole. In the present invention, the drug molecules themselves are interconnected into a polymer. Many small molecules which contain an acid and hydroxyl or amine groups can be polymerized by using this method.

Carbonyldiimidazole and thiocarbonyldiimidazole are best for synthesizing polyamide polymer, especially those molecules containing both amine and hydroxyl groups in the same molecules. The molecular weight of polyamide can be controlled by the reaction time and temperature.

Thionyl chloride-pyridine complexes or oxalyl chloride-pyridine complexes are best for synthesizing polyester. Pyridine used in this invention could be itself, or any other pyridine substituted derivatives. The molecular weights of the polyesters can be tuned by changing reaction time and reaction temperature.

Experiments have produced the following results:

### Preparation of Polysalicyclic Acid

### EXAMPLE 1

Salicylic acid (1.38 g, 0.01 mol) was dissolved in 10 ml dry acetonitrile. 1, 1′-Carbonyldiimidazole (1.60 g, 0.01mol) was added slowly by portions to the above solution. The reaction was brought to reflux temperature under the protection of nitrogen for 1 hour before 40 mg freshly-made sodium ethoxide was added. The reaction was kept refluxing for 5 days, cooled, evaporated to dryness Then 10 ml water was added, the solid was filtered and washed with plenty of water. The yield was 20%.

Product's IR data: 3111, 1747, 1728, 1604, 1486, 1452, 1246, 1045, 744 cm⁻¹.

### EXAMPLE 2

Salicylic acid (1.38 g, 0.01 mol), sodium salicylate (160 mg, 0.001 mol) was dissolved in 15 ml dry acetonitrile. 1, 1′-Carbonyldiimidazole (1.80 g, 0.01 mol) was added slowly by portions. The reaction was brought to reflux temperature under the protection of nitrogen for 5 days, cooled, and evaporated to dryness. Then 10 ml water was added, the solid was filtered and washed with plenty of water. The yield was 25%.

Product's IR data: 1745, 1730, 1600, 1479, 1454, 1247, 1045, 744 cm⁻¹.

### EXAMPLE 3

Salicylic acid (1.38 g, 0.01 mol) was dissolved in 10 ml dry acetonitrile. 1, 1′-Carbonyldiimidazole (1.62 g, 0.01 mol) was added slowly by portions to the above solution. The reaction was brought to reflux temperature under the protection of nitrogen for 1 hour before 40 mg freshly-made sodium methoxide was added. The reaction was kept refluxing for 5 days, cooled and evaporated to dryness. Then 10 ml water was added, the solid was filtered and washed with plenty of water. The yield was 30%.

Product's IR data: 3110, 1745, 1728, 1600, 1483, 1455, 1246, 1044, 748 cm⁻¹.

### EXAMPLE 4

Salicylic acid (5.6 g, 0.04 mol) was dissolved in 40 ml dry acetonitrile. 1, 1′-Carbonyldiimidazole (6.40 g, 0.04 mol) was added slowly by portions to the above solution. The reaction was brought to reflux temperature under the protection of nitrogen for 1 hour before 240 mg 98% concentrated H₂SO₄ was added. The reaction was kept refluxing for 2 days, cooled, evaporated to dryness. Then 10 ml water was added, the solid was filtered and washed with plenty of water. The yield was 60%.

Product's IR data: 3435, 1745, 1605, 1452, 1289, 1248, 1207, 748 cm⁻¹.

### EXAMPLE 5

Salicylic acid (1.38 g, 0.01 mol) was dissolved in 10 ml dry acetonitrile. 1,1′-Carbonyldiimidazole (1.60 g, 0.01 mol) was added slowly by portions to the above solution. The reaction was brought to reflux temperature under the protection of nitrogen for 1 hour before 120 mg 4-dimethylaminopyrinde was added. The reaction was kept refluxing for 2 days, cooled and evaporated to dryness. Then 10 ml water was added, the solid was filtered and washed with plenty of water. The yield was 50%.

Product's IR data: 3463, 1745, 1612, 1489, 1300, 1259, 1213, 1050, 757 cm⁻¹.

### EXAMPLE 6

1.30 g (0.01 mol) thionyl chloride was slowly added to 5 ml pyridine at room temperature. The resulting solution was stirred for 10 minutes. And then 1.37 g (0.01 mol) Salicyclic acid was added to the reaction mixture. The resulting mixture was stirred at room temperature for 12 hours. And then 10 ml water was added to quench the reaction. The resulting solid was washed with 10 ml water two times and was then dried under high vacuum at room temperature for 5 hours. 1.1 g poly salicyclic acid was obtained in 90% yield.

Product's IR data: 3462, 3078, 1745, 1606, 1581,1485, 1451, 1289, 1250, 1204, 1050, 746 cm⁻¹.

### EXAMPLE 7

1.37 g (0.01 mol) salicyclic acid was added to 5 ml pyridine at room temperature. The resulting solution was stirred for 10 minutes. And then 1.30 g (0.011 mol) thionyl chloride was slowly added to the reaction mixture. The resulting mixture was stirred at room temperature for 12 hours. And then 10 ml water was added to quench the reaction. The resulting solid was washed with 10 ml water two times and was then dried under high vacuum at room temperature for 5 hours. 1.1 g poly salicyclic acid was obtained in 90% yield.

Product's IR data: 3087, 1745, 1606, 1582, 1485, 1450, 1289, 1249, 1204, 1271, 1051, 747 cm⁻¹.

### EXAMPLE 8

1.30 g (0.011 mol) thionyl chloride was slowly added to 5 ml pyridine at room temperature. The resulting solution was stirred for 10 minutes. And then 1.37 g (0.01 mol) salicyclic acid was added to the reaction mixture. The resulting mixture was stirred at 70° C. for 6 hours. And then 10 ml water was added to quench the reaction. The resulting solid was washed with 10 ml water two times and was then dried under high vacuum at room temperature for 5 hours. 1.2 g poly salicyclic acid was obtained in 100% yield.

Product's IR data: 3083, 1743, 1605, 1580, 1486, 1450, 1289, 1250, 1207, 1123, 1050, 747 cm⁻¹.

### EXAMPLE 9

3.6 g 4-dimethyaminopyridine (DMAP) (0.03 mol) was dissolved in 10 ml dry acetonitrile. And then 1.30 g thionyl chloride (0.01 mol) was slowly added. The DMAP-SOCl₂ complex precipitated from the solution. And then 1.37 g salicyclic acid was added to the above mixture. The resulting mixture was stirred at room temperature for 12 hours, and then was evaporated to dryness. 10 ml water was added to quench the reaction. The resulting white solid was filtered and washed with 10 ml water 2 times, dried in high vacuum at room temperature for 5 hours. 1.2 g poly salicyclic acid was obtained in 100% yield.

Product's IR data: 1747, 1606, 1580, 1486, 1450, 1289, 1250, 1207, 1050, 745 cm⁻¹.

### EXAMPLE 10

1.2 g 4-dimethyaminopyridine (DMAP) (0.03 mol) was dissolved in 5 ml pyridine. And then 1.30 g thionyl chloride (0.011 mol) was slowly added. The DMAP-SOCl₂ complex precipitated from the solution. And then 1.37 g salicyclic acid was added to the above mixture. The resulting mixture was stirred at room temperature for 12 hours. And then 10 ml water was added to quench the reaction. The resulting white solid was filtered and washed with 10 ml water 2 times, dried in high vacuum at room temperature for 5 hours. 1.15 g poly salicyclic acid was obtained in 95% yield.

Product's IR data: 1743, 1604, 1579, 1486, 1450, 1289, 1250, 1204, 1050, 747 cm⁻¹.

### EXAMPLE 11

0.12 g 4-dimethyaminopyridine (DMAP) (0.001 mol) was dissolved in 5 ml pyridine. And then 1.30 g thionyl chloride (0.01 mol) was slowly added. The DMAP-SOCl₂ complex precipitated from the solution. And then 1.37 g salicyclic acid was added to the above mixture. The resulting mixture was stirred at room temperature for 12 hours. And then 10 ml water was added to quench the reaction. The resulting white solid was filtered and washed with 10 ml water 2 times, dried in high vacuum at room temperature for 5 hours. 1.1 g poly salicyclic acid was obtained in 90% yield.

Product's IR data: 1743, 1600, 1580, 1486, 1450, 1289, 1249, 1207, 1050, 746 cm⁻¹.

### EXAMPLE 12

1.38 g salicyclic acid (0.01 mol) was dissolved in 10 ml dry acetonitrile. Then 1.30 g thionyl chloride in 5 ml pyridine solution was added slowly. The resulting mixture was brought to reflux temperature for 12 hours, and then cooled and evaporated to dryness. 10 ml water was added to quench the reaction. The resulting solid was washed with 10 ml water two times and was then dried under high vacuum at room temperature for 5 hours. 1.1 g poly salicyclic acid was obtained in 90% yield.

Product's IR data: 1745, 1606, 1582, 1479, 1451, 1289, 1250, 1204, 1050, 747 cm⁻¹.

### Preparation of poly 4-aminobenzoic Acid

### EXAMPLE 13

1.37 g 4-aminobenzoic acid (0.01 mol) was dissolved in 20 ml acetonitrile. 1.62 g 1,1′-carbonyldiimidazole (0.01 mol) was slowly added to the above solution. The resulting solution was refluxing for 1 hour, and then the mixture was cooled, evaporated to remove acetonitrile. And then the residue was heated up to 170° C. for 30 minutes under the protection of nitrogen. Then immidazole was evaporated under high vacuum. 20 ml ethanol was added to the reaction mixture. And then 1 ml concentrated HCl solution was added slowly to quench the reaction. The reaction mixture was refluxed for 5 hours and was cool down to room temperature. Ethanol was evaporated and the resulting solid was washed with 10 ml water three times. The solid was dried under vacuum and 1.15 g of poly(4-aminobenzoic acid) was obtained as white powder. The yield was 95%.

Product's IR data: 3304, 1647, 1596, 1503, 1409, 1318, 1240, 1181, 849, 763 cm⁻¹.

### EXAMPLE 14

1.37 g 4-aminobenzoic acid (0.01 mol) was dissolved in 20 ml acetonitrile. 1. 62 g 1,1′-carbonyldiimidazol (0.01 mol) was slowly added to the above solution. The resulting solution was refluxing for 1 hour, and then the mixture was cooled, evaporated to remove acetonitrile. And then the residue was heated up to 170° C. for 30 minutes under the protection of nitrogen. 20 ml ethanol was added to the reaction mixture. And then 4 ml concentrated HCl solution was added slowly to quench the reaction. The reaction mixture was refluxed for 5 hours and was cool down to room temperature. Ethanol was evaporated and the resulting solid was washed with 10 ml water three times. The solid was dried under vacuum and 1.10 g of poly(4-aminobenzoic acid) was obtained as white powder. The yield was 90%.

Product's IR data: 3343, 1653, 1603, 1511, 1407, 1319, 1274, 845, 762 cm⁻¹.

### EXAMPLE 15

1.30 g thionyl chloride was slowly added to 5 ml pyridine at room temperature. The resulting solution was stirred for 10 minutes. And then the above solution was added slowly to 20 ml 4-aminobenzoic acid (1.38 g, 0.01 mol) acetonitrile solution. After the resulting mixture was stirred at room temperature for 12 hours, acetonitrile was evaporated. And then 10 ml water was added to quench the reaction. The resulting solid was washed with 10 ml water two times and was then dried under high vacuum at room temperature for 5 hours. 1.15 g poly 4-aminobenzoic acid was obtained in 95% yield.

Product's IR data: 3349, 1687, 1652, 1606, 1503, 1408, 1319, 1246, 1182, 852, 767 cm⁻¹.

### Preparation of poly4-aminosalicyclic Acid

### EXAMPLE 16

1.53 g 4-Aminosalicylic acid (0.01 mol) was dissolved in 20 ml acetonitrile. 1.62 g 1,1′-carbonyldiimidaz (0.01 mol) was slowly added to the above solution. The resulting solution was refluxing for 1 hour, and then the mixture was cooled, evaporated to remove acetonitrile. And then the residue was heated up to 170° C. for 30 minutes under the protection of nitrogen. The resulting mixture was cooled down to room temperature. And 20 ml ethanol was added. And then 4 ml concentrated HCl solution was added slowly to quench the reaction. The reaction mixture was refluxed for 5 hours and was cool down to room temperature. Ethanol was evaporated and the resulting solid was washed with 30 ml water. The solid was dried under vacuum and 1.3 g of poly(4-Aminosalicylic acid) was obtained as gray powder. The yield was 93%.

Product's IR data: 3434, 1628, 1610, 1380, 1275, 1181, 754, 690 cm⁻¹.

### EXAMPLE 17

1.57 g 4-aminosalicylic acid (0.01 mol) (0.01 mol) was dissolved in 20 ml acetonitrile. 1.62 g 1,1′-carbonyldiimidazole (0.01 mol) was slowly added to the above solution. The resulting solution was refluxing for 1 hour, and then the mixture was cooled, evaporated to remove acetonitrile. And then the residue was heated up to 170° C. for 30 minutes under the protection of nitrogen. Then immidazole was evaporated under high vacuum. 20 ml ethanol was added to the reaction mixture. And then 1 ml concentrated HCl solution was added slowly to quench the reaction. The reaction mixture was refluxed for 5 hours and was cool down to room temperature. Ethanol was evaporated and the resulting solid was washed with 10 ml water three times. The solid was dried under vacuum and 1.30 g of poly(4-Aminosalicylic acid) was obtained as white powder. The yield was 93%.

Product's IR data: 3315, 1658, 1603, 1546, 1229, 1181, 773, 687 cm⁻¹.

### EXAMPLE 18

1.30 g thionyl chloride was slowly added to 5 ml pyridine at room temperature. The resulting solution was stirred for 10 minutes. And then the above solution was added slowly to 20 ml 4-aminosalicyclic acid (1.57 g, 0.01 mol) acetonitrile solution. After the resulting mixture was stirred at room temperature for 12 hours, acetonitrile was evaporated. And then 10 ml water was added to quench the reaction. The resulting solid was washed with 10 ml water two times and was then dried under high vacuum at room temperature for 5 hours. 0.9 g poly 4-aminosalicyclic acid was obtained in 64% yield.

Product's IR data: 3446, 1756, 1668, 1611, 1558, 1317, 1237, 1158, 758 cm⁻¹.

### Preparation of Poly (2-aminobenzoic Acid)

### EXAMPLE 19

1.37 g 2-Aminobenzoic acid (0.01 mol) was dissolved in 20 ml acetonitrile. 1.62 g 1,1′-carbonyldiimidazol (0.01 mol) was slowly added to the above solution. The resulting solution was refluxing for 1 hour, and then the mixture was cooled, evaporated to remove acetonitrile. And then the residue was heated up to 170° C. for 30 minutes under the protection of nitrogen. The resulting mixture was cooled down to room temperature. And 20 ml ethanol was added. And then 4 ml concentrated HCl solution was added slowly to quench the reaction. The reaction mixture was refluxed for 5 hours and was cool down to room temperature. Ethanol was evaporated and the resulting solid was washed with 10 ml water three times. The solid was dried under vacuum and 1.1 g of poly(2-aminobenzoic acid) was obtained as white powder. The yield was 90%.

Product's IR data: 1661, 1610, 1585, 1424, 1371, 1304, 1063, 754, 662 cm⁻¹.

### EXAMPLE 20

1.30 g thionyl chloride was slowly added to 5 ml pyridine at room temperature. The resulting solution was stirred for 10 minutes. And then the above solution was added slowly to 20 ml 2-aminobenzoic acid (1.38 g, 0.01 mol) acetonitrile solution. After the resulting mixture was stirred at room temperature for 12 hours, acetonitrile was evaporated. And then 10 ml water was added to quench the reaction. The resulting solid was washed with 10 ml water three times. The solid was dried under vacuum and 1.1 g of poly(2-aminobenzoic acid) was obtained as white powder. The yield was 90%.

Product's IR data: 1751, 1663, 1607, 1549, 1482, 1305, 1237, 1162, 757 cm⁻¹.

All compounds were characterized by a proton nuclear magnetic resonance (NMR) spectroscopy and FT-Infrared (FT-IR). Infrared spectra were recorded on a Nicolet IR/44 spectrometer as KBr disks. ¹H NMR spectra were recorded in d₆-DMSO solution on a Bruker WM 360 (360-MHz) spectrometer with the solvent proton (DMSO) signal as an internal standard.

In application, the resulting synthesized polymers may be applied to medical devices where medicines are needed including the use of the resulting synthesized polymers in drug delivery implants. The resulting synthesized polymers may be applied for any of a variety of pharmaceuticals uses.

Synthetic polymer made by the methods described in this invention can be used to produce a variety of drug delivery products. They can be readily processed into pastes or solvent cast to yield films, coatings, microspheres and fibers with different geometric shapes for design of various medical implants, and may also be processed by compression molding and extrusion. Medical implant applications include the use of these polymers to form shaped articles such as vascular grafts and stents, bone plates, sutures, implantable sensors, implantable drug delivery devices, stents for tissue regeneration, and other articles that decompose harmlessly while delivering a selected low molecular weight drug at the site of implantation within a known time period. Drugs used in the present invention can also be incorporated into oral formulations and into products such as skin moisturizers, cleansers, pads, plasters, lotions, creams, gels, ointments, solutions, shampoos, tanning products and lipsticks for topical application.

The quantity of polymeric drug to be administered to a host which is effective for the selected use can be readily determined by those of ordinary skill in the art without undue experimentation. The quantity essentially corresponds stoichiometrically to the amount of drug which is known to produce an effective treatment for the selected use.

It is understood that the preceding description is given merely by way of illustration and not in limitation of the invention and that various modifications may be made thereto without departing from the spirit of the invention as claimed.

What is claimed is:

1. A method for making polymerized drugs in a one-step process, the method comprising:
condensing small drug molecules which contain an acid and an alcohol, or an thiol, or phenol or amine groups to polymerize them with a diimidazole reagent or thionyl chloride-pyridine complex, or oxalyl chloride-pyridine complex or any other similar acylating reagent to form a drug polymer which is a polyester or a polyamide in a one-step reaction.

2. The method of claim 1 comprising the single step of polymerizing salicylic acid with thionyl chloride-pyridine complexes to produce the polyester, poly(salicylic acid).

3. The method of claim 1 comprising the single step of polymerizing 4-amino-salicyclic acid with carbonyldiimidazole to produce the polyamide, poly(4-amino-salicyclic acid).

4. The method of claim 1 comprising the single step of condensing salicylic acid with thiocarbonyldiimidazole or other similar acylating reagents to produce the polymer poly(salicylic acid).

5. The method of claim 1 comprising the single step of condensing salicylic acid with thionyl chloride-pyridine complex, oxalyl chloride-pyridine complex, in which pyridine could be pyridine itself, or any other substituted pyridine derivatives.

6. The method of claim 1 comprising at least one acid group in the monomers.

7. The method of claim 6 wherein the acid group is a group taken from a list of acid groups including carboxylic acid, sulfonic acid, sulfinic acid, phosphonic acid, or any other acid group.

8. The method of claim 1 further comprising the step of applying the resulting synthesized polymers to medical devices.

9. The method of claim 1 further comprising the step of applying the resulting synthesized polymers in a drug delivery implant.

10. The method of claim 1 further comprising the step of applying the resulting synthesized polymers in pharmaceuticals uses.
